(19) United States
(12) Plant Patent Application Publication
(10) Pub. No.: US 2011/0010813 P1
(43) Pub. Date: Jan. 13, 2011

(54) Distinct cultivar of Hosta Plant named "Foxfire Palm Sunday"" which is suitable for landscape applications and container growing

(50) ;

(76) Inventor: Stephen John Schulte, Marshfield, WI (US)

Correspondence Address: Stephen J. Schulte, 28 Dalton Street, Asheville, NC, 28803, US

(21) Appl. No.: 12/459,948

(22) Filed: Jul. 10, 2009

(51) Int. Cl. A01H 5/00

(52) U.S. Cl. PLT/353

(57) ABSTRACT

A new cultivar of Hosta named ‘Foxfire Palm Sunday’, that is characterized by an intense, rubbery leaf texture, wide yellow/chartreuse leaf margins and unique, green center leaf pattern of irregular multi-lobular ‘palmate’ shape.

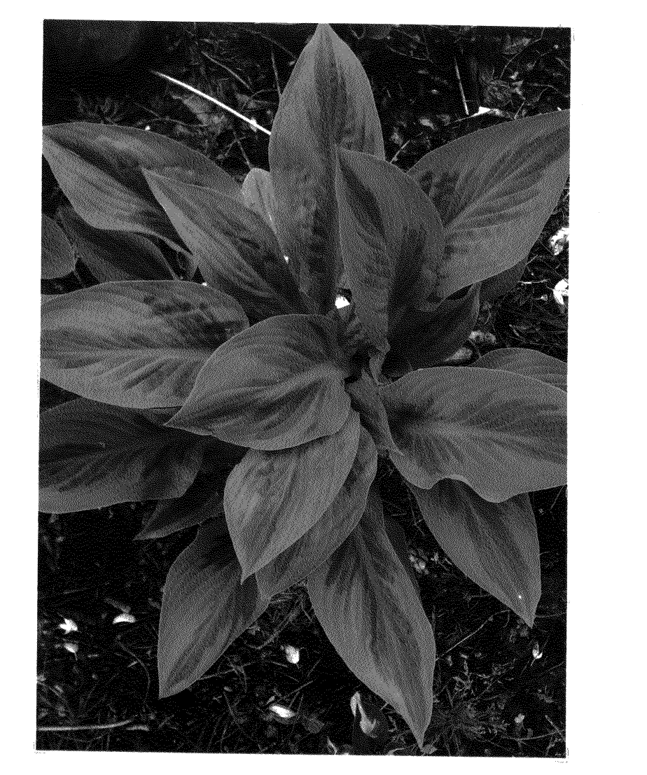

### CROSS REFERENCE TO RELATED APPLICATIONS

None

### STATEMENT REGARDING FEDERALLY SPONSERED R&D

None

### LATIN NAME OF GENUS AND SPECIES OF CLAIMED PLANT

Genus: Hosta
Species: Not routinely designated in this plant group as cross hybridizing often obscures the ‘species parentage’ and makes it impossible to clearly determine. Species designation may occur (always written in lower case italics) if the claimed plant were a direct mutation or selection of one of the thirty-three or more actual recognized species. The plant Hosta sieboldiana ‘Elegans’ would represent an example of this. This plant is a selection of a superior form of the species sieboldiana and is known in the market by its cultivar designation of ‘Elegans’. Any offspring resulting in a cross of sieboldiana ‘Elegans’ with another hosta though, would probably no longer carry the species designation. Such an offspring, as most all hostas, would simply be designated as Hosta ‘cultivar name’.

### VARIETY DENOMINATION

“Foxfire Palm Sunday”’ is the variety designation and the name that will be used in the landscape and container growing market trade.

### BACKGROUND OF THE INVENTION

The present invention relates to a new and distinct cultivar of Hosta plant, botanically known as Hosta hybrida, and hereinafter referred to by the cultivar name “Foxfire Palm Sunday”.

‘Foxfire Palm Sunday’ was selected in 2000 and originated from seedlings of an open pollination cross between pod parent H. ‘Independence Day’ (not patented) and an unknown pollen parent. The pod parent exhibits leaves with uniform gold margins and chartreuse centers. ‘Foxfire Palm Sunday’ was selected primarily for its unique foliage pattern, vigorous growth characteristics and intense ‘rubbery’ leaf texture. ‘Foxfire Palm Sunday’ differs from its pod parent in general shape, color and texture of the leaf as well as the unique ‘palm leaf’ variegation pattern within the leaf.

The inventor first accomplished asexual reproduction of the new cultivar by division in Marshfield, Wis. in 2007. Asexual reproduction of the new cultivar by division and tissue culture in 2008 has shown the unique features of ‘Foxfire Palm Sunday’ are stable and reproduced true to type in successive generations.

The new Hosta has not been observed under all possible environmental conditions. The appearance of the plant may vary somewhat with variations in environment such as degree of shading and ambient temperature but the genotype will remain stable.

### SUMMARY OF THE INVENTION

The following traits have been repeatedly observed and are determined to be the unique characteristics of ‘H. “Foxfire Palm Sunday”’ as a new and distinct cultivar.
1. Broad, irregular, yellow/chartreuse leaf margin.
2. Intense, rubbery leaf texture
3. Unique, green center leaf pattern of irregular multi-lobular ‘palmate’ shape

### BRIEF DESCRIPTION OF THE DRAWING

The accompanying color photograph illustrates the overall appearance of the new cultivar, showing the colors as true as it is reasonably possible to obtain in colored reproductions of this type. The photograph comprises a top perspective view of a plant “H. “Foxfire Palm Sunday”” in a cultivated area of the garden. There may be slight differences in the leaf colors of the photograph from the actual colors due to light reflectance.

### DETAILED BOTANICAL DESCRIPTION

In the following description, color references are made to The Royal Horticultural Society Colour chart except where general terms of ordinary dictionary significance are used. The following observations and measurements describe the plant as grown in the ground in Central Wisconsin under partial shade.
Botanical classification: Hosta hybrid cultivar “Foxfire Palm Sunday”.
Parentage: Pod parent Hosta “Independence Day”.
General description:
   a) Blooming period.—about four weeks from June 25^{th} to July 20^{th} in Wisconsin, USA.
   b) Plant habit.—Herbaceous perennial, clump forming, upright, vase shaped habit.
   d) Height and spread.—reaches about 24 cm (up to about 70 cm at maturity) in height and 70 cm (up to about 100 cm at maturity) in width.
   e) Hardiness.—USDA Zone 3-8.
   f) Culture.—Light shade to partial sun, moist soils of moderate fertility.
   g) Diseases and pests.—No susceptibility or resistance to diseases or other pests has been observed for “Foxfire Palm Sunday”.
   h) Root description.—Freely branched, fleshy.
   i) Propagation.—In vitro propagation is preferred using typical methods for Hosta, division is also possible.
   j) Root development.—Rooted transplants from tissue culture fully develop in a 72-cell liner in about 5-9 weeks in a greenhouse with average temperature of about 70 degrees F.
   k) Growth rate.—Vigorous.
Foliage description:
   a) Leaf shape.—Narrowly ovate.
   b) Leaf base.—tapered — elongated into petiole.
   c) Leaf size.—31 cm in length 17 cm in width.
   d) Leaf texture.—Very thick, near ‘rubbery’.
   e) Leaf quantity.—5-6 per shoot (eye).
   f) Leaf appearance.—moderately wavy in mature leaf, ‘crumpled’ in the juvenile form.
   g) Leaf surface.—top surface slightly shiny — bottom surface: glaucous bloom.
   h) Leaf venation.—About 10 to 12 pairs of veins, campylodrome pattern, impressed on upper surface and raised on lower surface, color matches foliage coloration. Margin exhibits notable ‘matrix’ veining between the primary veins.
   i) Leaf margin.—slightly wavy, extending well down into petiole as it narrows from a maximum width of 4 cm in the upper leaf to 0.5 cm in the petiole.
   j) Leaf arrangement.—Basal, radiate spirally from base.
   k) Leaf color.—(July 4^{th}, 50% shade, Wisconsin) Margin of the leaf is yellow green nearest to RHS 144A. Dark green center color (about RHS 137A) extends between the veins irregularly into the margin to produce the distinctively lobed ‘palmate’ variegation pattern. Margin assumes a deeper yellow color as season progresses and/or with greater exposure to sunshine.
   l) Petiole description.—a) About 15 cm in length, 2-3 cm in width b) Petiole color: Central region is green (about RHS 139C) with a yellow/green margin (about RHS 144A) c) Petiole surface: glaucous d) Petiole shape: Sulcate.
Flower scape description:
   a) Scape shape.—round, solid.
   b) Scape number.—One per mature eye.
   c) Scape posture.—Straight, upright no arching.
   d) Scape size.—45-75 cm long , 10-15 mm wide.
   e) Scape color.—green (about RHS 144A).
   f) Scape surface.—glaucous.
   g) Leaf bracts.—about 5-10 per scape, 4 cm length, 1.5 cm width, rigid, lanceolate in shape About RHS 136A in color, dull on upper surface, shiny on lower surface.
Flower type:
   a) Inflorescence type.—Terminal racemes of single tubular-shaped flowers, closely spaced on the last 12 cm of the scape.
   b) Lastingness of inflorescence.—about 4 weeks from first opening bloom to fading of last opening bloom, individual blooms lasting about one day.
   c) Flower number.—25-35.
   d) Flower fragrance.—none detected.
   e) Flower shape.—tubular.
   f) Flower bud shape.—Spathulate.
   g) Flower bud color.—Light lavender (about RHS 85B).
   h) Flower size.—6 cm long, 1.5 cm wide.
   i) Flower color.—White (about RHS N155B) with lavender (about RHS 85A) striations on the inner surface.
   j) Pedicel.—About 5 mm in length, 2 mm in diameter, RHS 145A.
   k) Perianth features.—Comprised of six tepals, 3 internal, 3 external, overlapping in expanded region and fused in tube region.
   l) Tepal shape.—Oblanceolate with acute apex.
   m) Tepal size.—6 cm in length and 1.5 cm in width.
   n) Tepal color.—White (about RHS N155B) with lavender (about RHS 85A) striations on the inner surface.
   o) Tepal texture.—Glaucous.
Reproductive organs:
Gynoecium: a) 1 pistil; style is about 4cm in length and 1 mm in diameter, white in color; stigma is 3 lobed and white in color; ovary is superior, compound, composed of 3 locules RHS 145A in color.
Androecium: 6 stamens; filament is about 4 cm in length and 1 mm in diameter, white in color, anthers are about 5 mm in length and 2 mm in width, RHS 199C in color. Pollen is abundant and RHS 15B in color.
Fruit: Not observed under growing conditions tested
Seeds Not observed under growing conditions tested.

I claim:

1. A new and distinct cultivar, named Hosta ‘H. “Foxfire Palm Sunday”’, substantially herein described and shown, characterized by an intense, rubbery leaf texture, wide yellow/chartreuse leaf margins and unique, green, center leaf pattern of irregular multi-lobular ‘palmate’ shape.
